(19) United States
(12) Patent Application Publication
(10) Pub. No.: US 2022/0362227 A1
(43) Pub. Date: Nov. 17, 2022

(54) COMPOSITION OF PHYTONUTRIENTS FOR DIABETES MANAGEMENT

(71) Applicant: ROSEMAN UNIVERSITY OF HEALTH SCIENCES, Las Vegas, CA (US)

(72) Inventors: Thuc T. LE, Las Vegas, NV (US); Yasuyo Urasaki, Las Vegas, NV (US)

(21) Appl. No.: 17/741,162

(22) Filed: May 10, 2022

Related U.S. Application Data
(60) Provisional application No. 63/187,330, filed on May 11, 2021.

(51) Int. Cl. A61K 31/4375; A61K 31/12; A61K 31/11; A61P 3/10

(52) U.S. Cl. CPC A61K 31/4375; A61K 31/12; A61K 31/11; A61P 3/10

(57) ABSTRACT

The present disclosure relates to compositions of phytonutrients and methods of treating diabetes by administering these compositions to subjects in need thereof. The compositions described herein are rationally designed compositions of phytonutrients that promote insulin sensitivity and have weight-loss potential. The compositions include formulation of rationally combined phytonutrients for diabetes treatment and management. Using nanofluidic proteomics, phytonutrients were classified as insulin sensitizing or anti-adipogenic based on their ability to enhance insulin-stimulated Akt2 phosphorylation and glucose uptake or inhibit adipogenesis, respectively. In some embodiments, the formulation includes a synergistic amount of insulin-sensitizing phytonutrients cinnamaldehyde and curcumin.

### CROSS-REFERENCE TO RELATED APPLICATIONS

This application claims the benefit of U.S. Provisional Patent Application 63/187,330, filed May 11, 2021.

### BACKGROUND

Diabetes mellitus is a group of metabolic disorders characterized by chronic high blood glucose condition [1,2]. Two main types of diabetes mellitus are Type 1 diabetes and Type 2 diabetes. In contrast, Type 1 diabetes affects approximately 5% of all patients diagnosed with diabetes. Type 1 diabetes accounts for up to 90% of diabetes in children and adolescents. Type 1 diabetes is a consequence of impaired insulin production due to a loss of pancreatic β cells. Type 1 diabetes is also known as insulin-dependent diabetes mellitus or juvenile diabetes. In contrast, Type 2 diabetes affects approximately 95% of all patients diagnosed with diabetes. Patients with Type 2 diabetes are mostly adults who experience insulin resistance, a condition in which cells of the body do not respond properly to insulin. Type 2 diabetes is commonly a consequence of overweight, smoking, and insufficient exercise. Type 2 diabetes is also known as non-insulin-dependent diabetes mellitus or adult-onset diabetes. Signs and symptoms of both types of diabetes mellitus include increased thirst, frequent urination, extreme hunger, fatigue, blurred vision, and frequent infections. Long-term complications of diabetes mellitus include cardiovascular disease, stroke, neuropathy, nephropathy, retinopathy, foot ulcer, and cognitive impairment. In 2018, approximately 10.5% of the US population, or 34.1 million people of all ages, had diabetes. The total cost for diabetes management in the US was estimated to be $327 billion in 2017.

Management of Type 1 diabetes requires insulin injection to lower blood glucose level [3]. In contrast, management of Type 2 diabetes involves weight management and glycemic control with lifestyle modifications and medications [4]. The first line of defense against Type 2 diabetes includes the maintenance of a healthy bodyweight via a healthy diet, regular physical exercise, and the cessation of smoking. In addition, anti-diabetes and anti-obesity medications can be prescribed. Anti-diabetes therapies using insulin, sulfonylurea and thiazolidinediones lower blood glucose level but lead to weight gain. Alternatively, anti-diabetes therapies using alpha-glucoside inhibitors, amylin mimetics, metformin, GLP-1R agonists, DPP-4 inhibitors and SGLT2 inhibitors are not associated with weight gain. Furthermore, a number of anti-obesity therapies are approved for weight management including orlistat, liraglutide, lorcaserin, phenteramine-topiramate ER, and naltrexone-bupropion XR. However, current anti-obesity and anti-diabetes therapies are frequently associated with adverse side effects, which limit their long-term usage for weight management and glycemic control [5].

In the management of diabetes, insulin therapy is positively correlated with glycemic control and weight gain. Weight gain in diabetic patients is associated with hyperlipidemia, hypertension, and deterioration of β-cell function. Thus, any insulin therapy or diabetic medication that can provide glycemic control without weight gain is highly desirable. Many approved anti-diabetic pharmaceutical agents are associated with adverse effects, which limit their long-term usage for diabetes care. Thus, there is an unmet need for formulations that can effectively manage diabetes without causing weight gain.

### BRIEF DESCRIPTION OF THE DRAWINGS

FIG. 1A identifies Akt phosphoisoform 1 (Akt1) using a capillary isoelectric focusing (cIEF) immunoassay, as shown by a cIEF electropherogram.

FIG. 1B identifies Akt phosphoisoform 2 (Akt2) by a cIEF electropherogram.

FIG. 1C identifies Akt phosphoisoform 3 (Akt3) by a cIEF electropherogram.

FIG. 1D identifies pan-Akt or all three Akt1-3 isoforms by a cIEF electropherogram. The letter p represents the number of phosphorylation of Akt isoforms. Peaks on cIEF electropherograms are labeled Akt1, Akt2, and Akt3.

FIG. 1E shows percentage of Akt1, Akt2, and Akt3 as a function of all Akt isoforms.

FIG. 1F shows a cIEF electropherogram of pAkt (Thr308). Total cell extracts (TCE) from human primary preadipocytes were used for cIEF immunoassays.

FIG. 1G shows a cIEF electropherogram of pAkt (Thr450). Dashed line highlights the even distribution of pAkt (Thr450) peaks at low and high pI values. TCE were as for FIG. 1F.

FIG. 1H shows a cIEF electropherogram of pAkt (S473). TCE were as for FIG. 1F.

FIG. 2A shows insulin promotes phosphorylation of Akt isoforms, by a cIEF electropherogram of Akt1 following treatment with insulin for 30 minutes. Arrows point to new peaks, which appear after treatment with insulin.

FIG. 2B shows insulin promotes phosphorylation of Akt isoforms, by a cIEF electropherogram of Akt2, following the protocol of FIG. 2A.

FIG. 2C shows insulin promotes phosphorylation of Akt isoforms, by a cIEF electropherogram of Akt3, following the protocol of FIG. 2A.

FIG. 2D shows insulin promotes phosphorylation of Akt isoforms, by a cIEF electropherogram of pAkt (Thr308), following the protocol of FIG. 2A.

FIG. 2E shows insulin promotes phosphorylation of Akt isoforms, by a cIEF electropherogram of pAkt (Thr450), following the protocol of FIG. 2A. Dashed line highlights the uneven distribution of pAkt (Thr450) peaks following treatment insulin, where peaks at low pI values were greater than those at high pI values.

FIG. 2F shows insulin promotes phosphorylation of Akt isoforms, by a cIEF electropherogram of pAkt (Ser473), following the protocol of FIG. 2A

FIG. 2G shows capillary Western (CW) immunoassays of pAkt (Thr308), pAkt (Thr450), and pAkt (Ser473) before and after treatment with insulin. Pan-Akt and β-actin served as the loading controls.

FIG. 2H shows relative expression level of pAkt (Thr308), pAkt (Thr450) and pAkt (Ser473) before (solid black columns, normalized to 1) and after (textured) treatment with insulin. Error bars indicate standard deviations across six repeated measurements using CW immunoassays per experimental condition. Asterisks indicate a statistical significance of p≤0.05 versus untreated control.

FIG. 3A shows a cIEF electropherogram of Akt1, following treatment with cinnamaldehyde for 30 minutes.

FIG. 3B shows a cIEF electropherogram of Akt2, following treatment with cinnamaldehyde for 30 minutes. Arrow points to a new peak at pI 5.41, which appears following treatment with cinnamaldehyde.

FIG. 3C shows a cIEF electropherogram of Akt3, following treatment with cinnamaldehyde for 30 minutes.

FIG. 3D shows a cIEF electropherogram of pAkt (Thr308), following treatment with cinnamaldehyde for 30 minutes.

FIG. 3E shows a cIEF electropherogram of pAkt (Thr450), following treatment with cinnamaldehyde for 30 minutes. Dashed line highlights the uneven distribution of pAkt (Thr450) peaks following treatment with cinnamaldehyde, where peaks at low pI values constitute greater percentage of total pAkt (Thr450) compared to peaks at high pI values.

FIG. 3F shows a cIEF electropherogram of pAkt (Ser473).

FIG. 3G shows CW immunoassays of pAkt (Thr308), pAkt (Thr450), and pAkt (Ser473) before and after treatment with cinnamaldehyde. Pan-Akt and β-actin served as the loading controls.

FIG. 3H shows relative expression level of pAkt (Thr308), pAkt (Thr450) and pAkt (Ser473) before (solid black columns, normalized to 1) and after (textured columns) treatment with cinnamaldehyde. Error bars indicate standard deviations across six repeated measurements using CW immunoassays per experimental condition. Asterisks indicate a statistical significance of p≤0.05 versus untreated control.

FIG. 4A shows a cIEF electropherogram of Akt1 following insulin treatment alone (upper panel) or insulin and cinnamaldehyde treatment (lower panel).

FIG. 4B shows a cIEF electropherogram of Akt2 following insulin treatment alone (upper panel) or insulin and cinnamaldehyde treatment (lower panel). Arrows point to pAkt2 (Ser473) peaks, which increase following insulin and cinnamaldehyde treatment (lower panel) compared to insulin treatment alone.

FIG. 4C shows a cIEF electropherogram of Akt3 following insulin treatment alone (upper panel) or insulin and cinnamaldehyde treatment (lower panel).

FIG. 4D shows cinnamaldehyde promotes insulin-stimulated phosphorylation of pAkt (S473), as demonstrated by cIEF electropherogram. Upper panel: insulin treatment alone. Lower panel: insulin and cinnamaldehyde treatment.

FIG. 4E shows CW immunoassays of p-Akt (S473) expression level following insulin treatment alone (third lane, first row) or insulin and cinnamaldehyde treatment (fourth lane, first row). Pan-Akt (first and second lanes, first row) and β-actin (second row) served as the loading controls.

FIG. 4F shows relative expression level of pAkt2 (Ser473) and pAkt (Ser473) following treatment with insulin alone (solid black) or treatment with insulin together with cinnamaldehyde (textured). Only cIEF immunoassay was used for the quantitation of pAkt2 (Ser473) expression level. Both cIEF and CW immunoassays were used for the quantitation of pAkt (S473) expression level. Error bars indicate standard deviations across six repeated measurements per experimental condition. Asterisks indicate a statistical significance of p≤0.05 versus treatment with insulin alone.

FIG. 5A shows a cIEF electropherogram of Akt2 in preadipocytes: untreated control.

FIG. 5B shows a cIEF electropherogram of Akt2 in preadipocytes treated with cinnamaldehyde. Arrows point to new peaks, which appear after treatment versus untreated control.

FIG. 5C shows a cIEF electropherogram of Akt2 in preadipocytes treated with curcumin. Arrows point to new peaks, which appear after treatment versus untreated control.

FIG. 5D shows a cIEF electropherogram of Akt2 in preadipocytes treated with cinnamaldehyde and curcumin. Arrows point to new peaks, which appear after treatment versus untreated control, showing curcumin synergizes with cinnamaldehyde to promote Akt2 phosphorylation.

FIG. 5E shows a cIEF electropherogram of Akt2 in preadipocytes treated with insulin alone. Arrows point to new peaks, which appear after treatment versus untreated control.

FIG. 5F shows a cIEF electropherogram of Akt2 in preadipocytes treated with insulin and cinnamaldehyde. Arrows point to new peaks, which appear after treatment versus untreated control.

FIG. 5G shows a cIEF electropherogram of Akt2 in preadipocytes treated with insulin and curcumin. Arrows point to new peaks, which appear after treatment versus untreated control.

FIG. 5H shows a cIEF electropherogram of Akt2 in preadipocytes treated with insulin, cinnamaldehyde, and curcumin. Arrows point to new peaks, which appear after treatment versus untreated control, showing curcumin synergizes with cinnamaldehyde to promote Akt2 phosphorylation.

FIG. 6A shows curcumin and cinnamaldehyde synergize to enhance insulin-stimulated Akt2 phosphorylation and glucose uptake, by quantitative analyses of cIEF data presented in FIGS. 5A-5H on the effects of cinnamaldehyde and curcumin on insulin-stimulated pAkt2 (Ser473) phosphorylation. Error bars indicate standard deviations across six repeated measurements per experimental condition.

FIG. 6B shows flow cytometry analyses of glucose uptake in preadipocytes treated with insulin alone or treated with insulin and cinnamaldehyde.

FIG. 6C) shows flow cytometry analyses of glucose uptake in preadipocytes treated with insulin alone or treated with insulin and curcumin).

FIG. 6D shows flow cytometry analyses of glucose uptake in preadipocytes treated with insulin alone or treated with insulin and cinnamaldehyde and curcumin).

FIG. 6E shows mean fluorescence intensity as a measure of glucose uptake following treatment of preadipocytes with insulin alone or insulin together with phytonutrients. Error bars indicate standard error of the mean (SEM) of 30,000 cells analyzed per experimental condition. Asterisks indicate a statistical significance of p≤0.05 versus treatment with insulin alone (*) or versus treatment with insulin together with cinnamaldehyde (**).

FIG. 7A shows a cIEF electropherogram of Akt2 in preadipocytes: untreated control.

FIG. 7B shows a cIEF electropherogram of Akt2 in preadipocytes treated with berberine.

FIG. 7C shows a cIEF electropherogram of Akt2 in preadipocytes treated with insulin. Arrows point to new peaks that appeared after treatment versus untreated control.

FIG. 7D shows a cIEF electropherogram of Akt2 in preadipocytes treated with insulin and berberine. Arrows point to new peaks that appeared after treatment versus untreated control. Comparison to FIG. 7C shows berberine is a non-insulin sensitizing phytonutrient.

FIG. 7E shows a cIEF electropherogram of Akt2 in preadipocytes treated with cinnamaldehyde, curcumin, and insulin. Arrows point to new peaks that appeared after treatment versus untreated control.

FIG. 7F shows a cIEF electropherogram of Akt2 in preadipocytes treated with cinnamaldehyde, curcumin, berberine, and insulin. Arrows point to new peaks that appeared after treatment versus untreated control. Comparison to FIG. 7E shows berberine is a non-insulin sensitizing phytonutrient.

FIG. 8A shows berberine has no effect on insulin-stimulated Akt2 phosphorylation, as demonstrated by quantitative analyses of cIEF data presented in FIG. 7A-7F on the effects of berberine on insulin-stimulated pAkt2 (Ser473) phosphorylation. F2 is a combination of cinnamaldehyde, curcumin, and berberine. Error bars indicate standard deviations across six repeated measurements per experimental condition.

FIG. 8B shows a flow cytometry analysis of glucose uptake in preadipocytes treated with insulin alone or treated with insulin and berberine.

FIG. 8C shows a flow cytometry analysis of glucose uptake in preadipocytes treated with insulin alone or treated with insulin, cinnamaldehyde and curcumin.

FIG. 8D shows a flow cytometry analysis of glucose uptake in preadipocytes treated with insulin alone or treated with insulin, cinnamaldehyde, curcumin, and berberine.

FIG. 8E shows mean fluorescence intensity as a measure of glucose uptake following treatment of preadipocytes with insulin alone or insulin together with phytonutrients. Error bars indicate standard error of the mean (SEM) of 30,000 cells analyzed per experimental condition. Asterisks indicate a statistical significance of p≤0.05 versus treatment with insulin alone. FIG. 8E shows berberine has no effect on insulin-stimulated glucose uptake.

FIG. 9A shows berberine inhibits adipogenesis in primary human preadipocytes, as shown by Oil Red O staining of intracellular lipid droplets in undifferentiated preadipocytes (first panel, upper row), differentiated adipocytes (second panel, upper row), differentiated adipocytes treated with cinnamaldehyde (third panel, upper row), differentiated adipocytes treated with curcumin (first panel, lower row), differentiated adipocytes treated with berberine (second panel, lower row), and differentiated adipocytes treated with a F2 formulation comprising cinnamaldehyde, curcumin, and berberine (third panel, lower row). Oil Red O staining was performed at day 14^{th} post-differentiation.

FIG. 9B show capillary Western immunoassays to evaluate the expression of adipogenic biomarkers PPARy (first row), FASN (second row), and PLIN1 (third row) in undifferentiated preadipocytes (first column), differentiating adipocytes (second column), differentiating adipocytes treated with cinnamaldehyde (third column), differentiating adipocytes treated with curcumin (fourth column), differentiating adipocytes treated with berberine (fifth column), and differentiating adipocytes treated with a F2 formulation comprising cinnamaldehyde, curcumin, and berberine (sixth column). TCE used for CW immunoassays were collected at sixth day post-differentiation. β-actin (fourth row) served as a loading control.

FIG. 9C shows quantitative analyses of CW immunoassay data. Error bars indicate standard deviations across six repeated measurements per experimental condition. Asterisks indicate a statistical significance of p≤0.05 versus undifferentiated preadipocytes (*) or versus differentiating adipocytes (**).

FIG. 10A shows F2 reduces weight gain in diet-induced obesity (DIO) mice, as demonstrated by average bodyweight as a function of time on specified diets of three group of mice: lean diet, high-fat diet, and high-fat diet supplemented with F2.

FIG. 10B shows a representative photo of mice from each diet group shown in FIG. 10A at 17th week.

FIG. 10C shows average bodyweight at 17^{th} week as a function of mice group on specified diets. LD: lean diet; HFD: high-fat diet; HFD+F2: high-fat diet supplemented with F2.

FIG. 10D shows average rate of weight gain in grams per week (g/w) as a function of mice group on specified diets LD, HFD, and HFD+F2. The error bars indicate the standard deviations of 40 mice per animal group. The asterisks indicate a statistical significance of p≤0.05 versus the lean diet group (*) or versus the high-fat diet group (**).

FIG. 11A shows F2 improves glucose tolerance in DIO mice, as demonstrated by blood glucose level as a function of time post injection.

FIG. 11B shows fold change in blood glucose level as a function of time post injection.

FIG. 11C shows F2 reduces HbAlc in DIO mice, as shown by HbAlc (%). LD: lean diet; HFD: high-fat diet; HFD+F2: high-fat diet supplemented with F2. The error bars indicate the standard deviations of 40 mice per animal group. The asterisks indicate a statistical significance of p≤0.05 versus the lean diet group (*) or versus the high-fat diet group (**).

FIG. 11D shows F2 reduces HbAlc in DIO mice, as shown by HbAlc (mmol/mol) as a function of animal groups. LD: lean diet; HFD: high-fat diet; HFD+F2: high-fat diet supplemented with F2. Statistical measures were as for FIG. 11C.

FIG. 12A shows F2 reduces blood lipids in DIO mice, specifically, triglyceride, as a function of animal group on specified diets. LD: lean diet; HFD: high-fat diet; HFD+F2: high-fat diet supplemented with F2. Blood samples terminally collected after 17 weeks on specified diets were used for measurement. The error bars indicate the standard deviations of 40 mice per animal group. The asterisks indicate a statistical significance of p≤0.05 versus the lean diet group (*) or versus the high-fat diet group (**).

FIG. 12B shows F2 reduces blood lipids in DIO mice, specifically, cholesterol. Diets and statistical measures were as for FIG. 12A.

FIG. 12C shows F2 reduces blood lipids in DIO mice, specifically, high-density lipoprotein (HDL). Diets and statistical measures were as for FIG. 12A.

FIG. 12D shows F2 reduces blood lipids in DIO mice, specifically, low-density lipoprotein (LDL). Diets and statistical measures were as for FIG. 12A.

FIG. 13A shows F2 reduces visceral adiposity and liver steatosis in DIO mice, as demonstrated by H&E histology of visceral adipose tissues collected from three animal groups on specified diets. LD: lean diet; HFD: high-fat diet; HFD+F2: high-fat diet supplemented with F2.

FIG. 13B shows F2 reduces visceral adiposity and liver steatosis in DIO mice, as demonstrated by H&E histology of liver tissues collected from three animal groups on the specified diets used in FIG. 13A.

FIG. 13C shows visceral adipose tissue weight as a function of animal groups on the specified diets used in FIG. 13A. Visceral adipose tissues were terminally collected after 17 weeks on specified diets. The error bars indicate the standard deviations of 40 mice per animal group. The asterisks indicate a statistical significance of p≤0.05 versus the lean diet group (*) or versus the high-fat diet group (**).

FIG. 13D shows liver tissue weight as a function of animal groups on the specified diets used in FIG. 13A. Liver tissues were terminally collected after 17 weeks on specified diets. Statistical measures were as for FIG. 13C.

### DETAILED DESCRIPTION

Herein, we describe a novel approach for rational combination of phytonutrients for diabetes management. Anti-diabetic phytonutrients, or natural compounds found in plants, are generally recognized as safe for long-term diabetes care. Phytonutrients in plants and mushrooms have anti-obesity and anti-diabetes effects by modulating physiological pathways that regulate appetite, metabolism, lipid absorption, insulin sensitivity, thermogenesis, and gut microbiota [6]. Consumption of phytonutrients is generally considered as a safe, widely available and inexpensive approach to manage obesity and diabetes. The disclosure herein identifies phytonutrients and combinations thereof that promote insulin sensitivity and have weight-loss potentials.

The present disclosure relates to novel compositions of phytonutrients and methods of treating diabetes by administering these compositions to subjects in need thereof. The compositions described herein are rationally designed compositions of phytonutrients that promote insulin sensitivity and have weight-loss potential.

The compositions include formulation of rationally combined phytonutrients for diabetes treatment and management. Using nanofluidic proteomics, phytonutrients were classified as insulin sensitizing or anti-adipogenic based on their ability to enhance insulin-stimulated Akt2 phosphorylation and glucose uptake or inhibit adipogenesis, respectively. In some embodiments, the formulation includes a synergistic amount of insulin-sensitizing phytonutrients cinnamaldehyde and curcumin.

The formulation of rationally combined phytonutrients exhibit unique properties of providing glycemic control without weight gain that are distinct from those of individual phytonutrients. The formulations of rationally combined phytonutrients disclosed herein may improve glucose tolerance, reduce HbAlc, prevent weight gain, reduce blood lipid level, and/or suppress liver steatosis.

In one aspect, the compositions disclosed herein may include any combination of phytonutrients cinnamaldehyde, curcumin, and berberine. For example, the combination of phytonutrients may be (1) cinnamaldehyde and curcumin; (2) cinnamaldehyde and berberine; (3) curcumin and berberine; or cinnamaldehyde, curcumin, and berberine. In a particular embodiment, the composition may include a synergistic amount of cinnamaldehyde and curcumin, and additionally include an effective amount of berberine.

In another aspect, the invention relates to methods of treating or managing diabetes comprising administering a therapeutically effective amount of the compositions disclosed herein. In some embodiments, diabetes is treated with minimal or no weight gain by the subject.

The compositions disclosed herein may further improve glucose tolerance, reduce HbAlc, prevent weight gain, reduce blood lipid level, and/or suppress liver steatosis.

### EXAMPLES

### Example 1: Classification of Phytonutrients as Insulin Sensitizing or Anti-Adipogenic

Using nanofluidic proteomics, phytonutrients were screened and classified as either insulin sensitizing or anti-adipogenic. Phytonutrients cinnamaldehyde and curcumin were identified as insulin-sensitizing phytonutrients by their ability to promote pAkt2 (Thr450) phosphorylation, enhance insulin-stimulated pAkt2 (Ser473) phosphorylation, and increase glucose uptake in primary human preadipocytes. In addition, nine phytonutrients were identified, including berberine, as non-insulin sensitizing and anti-adipogenic phytonutrients. The combination of insulin-sensitizing and anti-adipogenic phytonutrients comprising cinnamaldehyde, curcumin, and berberine enhanced insulin-stimulated glucose uptake and inhibited adipogenesis in human primary preadipocytes. This combination was named the F2 formulation. In an animal model of diet-induced obesity and diabetes, the data shows that dietary supplementation with F2 formulation improved glucose tolerance, reduced HbAlc, prevented weight gain, reduced blood lipid level, and suppressed liver steatosis. Collectively, the proteomic method disclosed herein was used to identify insulin-sensitizing phytonutrients and a platform for the rational combinations of insulin-sensitizing and anti-diabetic phytonutrients. Furthermore, the therapeutic potential for the F2 formulation to provide glycemic control without weight gain for long-term diabetes care was demonstrated.

Insulin-Sensitizing Phytonutrients. The serine/threonine protein kinase B, also known as Akt, regulates many critical cellular processes including the canonical insulin-signaling cascade of IR/IRS/PI3K/Akt [7]. The Akt kinase family comprises three highly homologous isoforms of Akt1, Akt2 and Akt3, which exhibit distinctive functional specificity and tissue distribution [8]. Whereas Akt1 regulates cell growth, survival, and migration; Akt2 regulates glucose metabolism; and Akt3 regulates cell volume and ion homeostasis and neuronal development. Using a patented nanofluidic proteomics platform, phytonutrients were screened for their effects on the post-translational modifications of Akt2. Two phytonutrients were identified, cinnamaldehyde and curcumin, as insulin sensitizers. Both cinnamaldehyde and curcumin promoted Akt2 phosphorylation and glucose uptake in primary human preadipocytes. The experimental approaches are described below.

Mapping Akt Isoforms Using Capillary Isoelectric Focusing (cIEF) Immunoassays. cIEF immunoassays separate proteins in total cell extracts (TCEs) by their isoelectric points (pIs) in nano-capillaries. The positions of the proteins are stabilized via UV-irradiated crosslinking of proteins to the sidewalls of the capillaries. A primary antibody that recognizes a specific protein of interest is introduced to individual capillaries. Then a secondary antibody linked to horseradish peroxidase is introduced. Following the introduction of the chemiluminescent substrates, the distribution of the isoforms of a protein is detected and presented graphically as electropherograms of intensity versus isoelectric points. Phosphorylation and acetylation of a protein generally leads to shifts toward lower pI values. In contrast, glycosylation of a protein generally leads to a shift toward higher pI values.

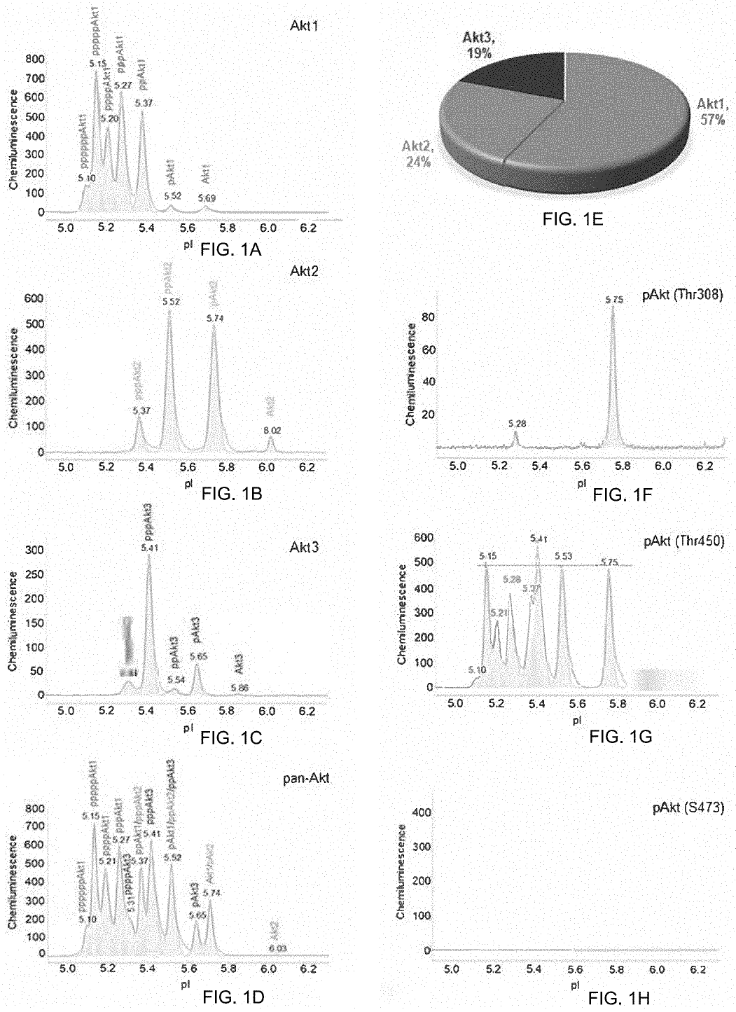

Identification of Akt Isoforms on cIEF Electropherograms. Using primary antibodies specific to Akt1, Akt2, or Akt3, these Akt isoforms can be identified in individual nano-capillaries. FIG. 1A identifies the distribution of an unphosphorylated Akt1 isoform and various Akt1 phosphoisoforms in primary human preadipocytes. FIG. 1B identifies the distribution of an unphosphorylated Akt2 isoform and various Akt2 phosphoisoforms in human primary preadipocytes. FIG. 1C identifies the distribution of an unphosphorylated Akt3 isoform and various Akt3 phosphoisoforms in primary human preadipocytes. In addition, using a primary antibody that recognizes all Akt isoforms (pan-Akt), the relative concentrations of each Akt isoforms as a function of total Akt can be directly compared and quantified (FIG. 1D). In human primary preadipocytes, Akt1, Akt2, and Akt3 isoforms constitute 57%, 24%, and 19%, respectively (FIG. 1F). The composition of Akt isoforms varies as a function of tissue types [9]. For example, Akt3 is the major isoform in human neuronal cells, accounting for 50% of all Akt isoforms. In contrast, liver cells, T cells, and intestinal cells do not express Akt3 isoform. Furthermore, phosphorylated amino acid residues can be identified using primary antibodies that recognize specific phosphorylation sites on Akt. However, phosphorylated amino acid residues detected herein are of all Akt isoforms because primary antibodies cannot distinguish phosphorylated amino acid residues of individual Akt isoforms. Interestingly, pAkt (Thr308) (FIG. 1F) and pAkt (Thr450) (FIG. 1G) are present and pAkt (S473) is absent in human primary preadipocytes. On the one hand, pAkt (Thr308) and Akt2 overlap significantly at pI 5.75, which suggests that most of pAkt (Thr308) is attributable to pAkt2 (Thr308). On the other hand, pAkt (Thr450) overlaps with all of Akt1, Akt2, and Akt3 at multiple pIs, which suggests that Thr450 residue is constitutively phosphorylated in all Akt isoforms. Notably, pAkt (Thr450) peaks at low and high pI values are evenly distributed in human primary preadipocytes (FIG. 1G, dashed line).

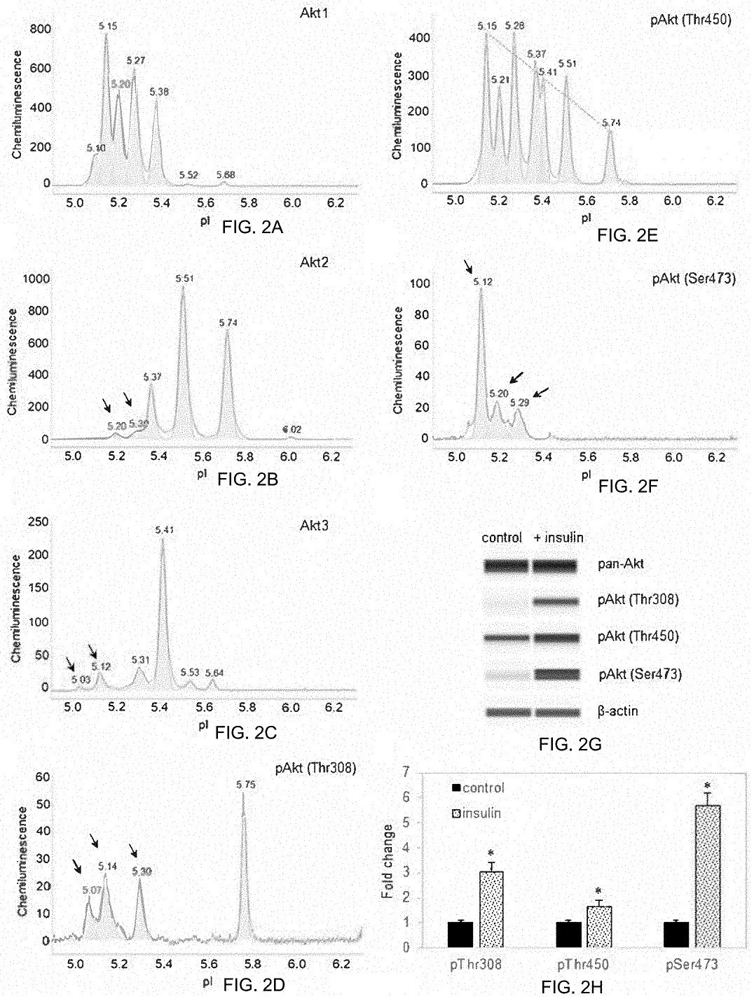

Insulin Promotes Akt Phosphorylation. Following the treatment of human primary preadipocytes with 100 nM insulin for 30 minutes, cIEF electropherograms of Akt isoforms and phosphorylated isoforms were examined and compared to those without insulin treatment. Surprisingly, the cIEF electropherograms of Akt1 isoform were nearly identical before and after insulin treatment (FIG. 2A). Expectedly, insulin treatment increased Akt2 phosphorylation as indicated by two new peaks at pI 5.20 and pI 5.30 on the Akt2 electropherograms (FIG. 2B). Similarly, insulin treatment also increased Akt3 phosphorylation as indicated by two new peaks at pI 5.03 and pI 5.12 on the Akt3 electropherograms (FIG. 2C). Insulin treatment increased the phosphorylation of pAkt (Thr308) as indicated by three new peaks at pI 5.07, pI 5.14, and pI 5.30 (FIG. 2D). Notably, the distribution of pAkt (Thr450) peaks shifted toward those at the lower pI values following insulin treatment, which indicated an increase in pAkt (Thr450) phosphorylation (FIG. 2E). Furthermore, three peaks at pI 5.12, pI 5.20, and pI 5.29 were detected for pAkt (Ser473) following treatment with insulin (FIG. 2F). Increases in peaks at pI 5.12 and lower pI values for pAkt (Thr308), pAkt (Thr450), and pAkt (Ser473) following insulin treatment indicated that insulin also promoted phosphorylation of Akt1 isoform. However, due to the highly phosphorylated states of Akt1 isoform before insulin treatment, it was plausible that insulin-stimulated Akt1 phosphorylation was not detectable on cIEF electropherograms. Using an alternative measurement method, capillary Western immunoassays, insulin-stimulated phosphorylation of pAkt (Thr308), pAkt (Thr450), and pAkt (Ser473) were also detected (FIG. 2G). Quantitative densitometry analyses revealed insulin-stimulated increases of approximately 3, 1.6, and 5.7 folds for pAkt (Thr308), pAkt (Thr450), and pAkt (Ser473), respectively (FIG. 211).

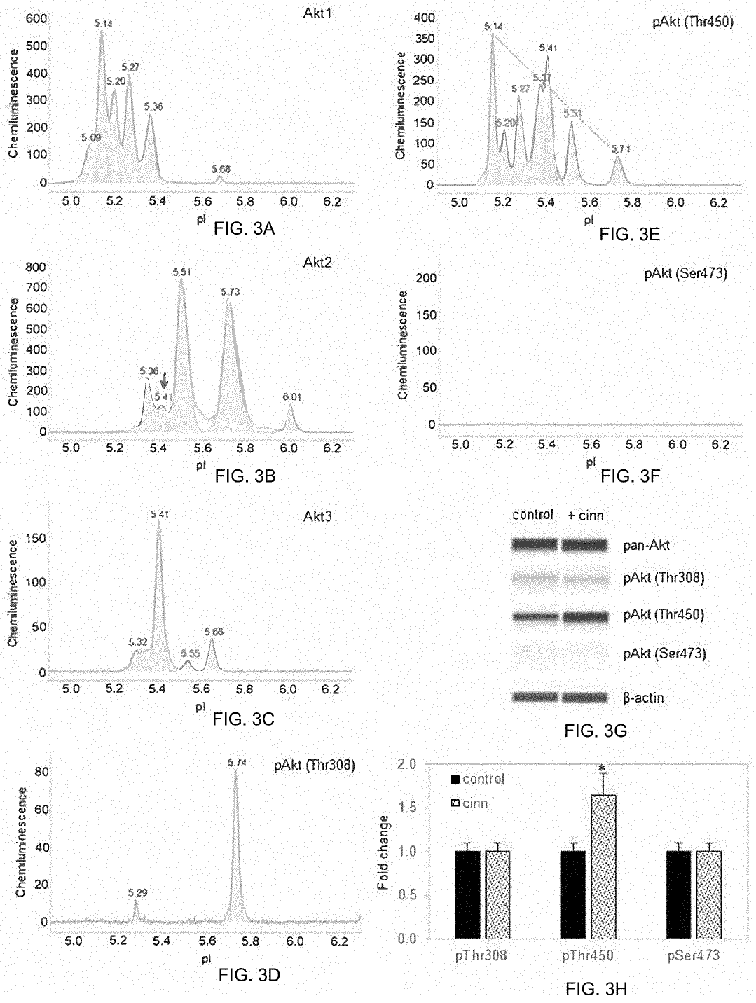

Cinnamaldehyde Selectively Promotes pAkt (Thr450) Phosphorylation. Following a screen of over 50 phytonutrients, cinnamaldehyde, was identified as an insulin sensitizer. Cinnamaldehyde is a principal constituent of cinnamon bark essential oil. Cinnamaldehyde gives cinnamon its flavor and aroma. Treatment of human primary preadipocytes with cinnamaldehyde had no observable effect on the electropherogram of Akt1 or Akt3 isoforms (FIGS. 3A and 3C). Interestingly, cinnamaldehyde treatment induced a new peak at pI 5.41 on the Akt2 electropherogram (FIG. 3B). Cinnamaldehyde treatment had no effect on the expression of pAkt (Thr308) or pAkt (Ser473) in preadipocytes (FIGS. 3D and 3F). However, cinnamaldehyde treatment induced a shift in the distribution of pAkt (Thr450) peaks toward those at lower pI values (FIG. 3E). The effects of cinnamaldehyde treatment on the pAkt (Thr450) electropherogram was nearly identical to that of insulin treatment, suggesting a promotion of pAkt (Thr450) phosphorylation. Measurements using capillary Western immunoassays further confirm the specific positive effects of cinnamaldehyde treatment on the phosphorylation of pAkt (Thr450) (FIG. 3G). Cinnamaldehyde treatment increased pAkt (Thr450) phosphorylation by an average of 1.6 folds but had no effect on the phosphorylation of pAkt (Thr308) or pAkt (Ser473) (FIG. 311).

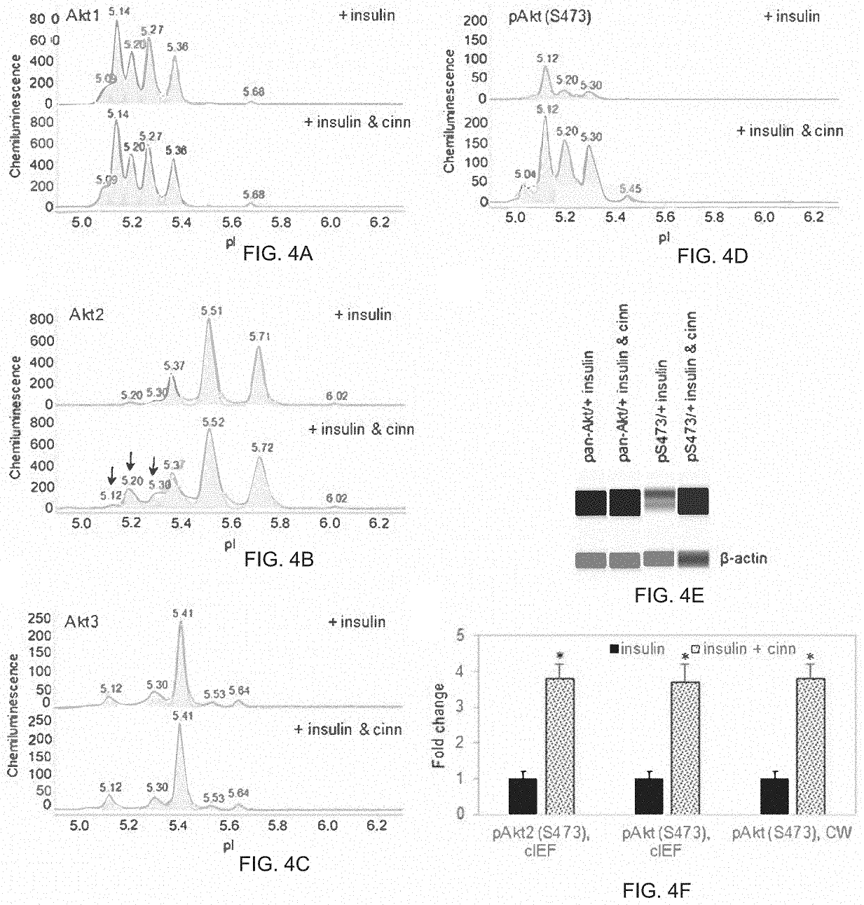

Cinnamaldehyde Promotes Insulin-Stimulated pAkt2 (Ser473) Phosphorylation. Treatment of human primary preadipocytes with insulin together with cinnamaldehyde had no observable effect on the phosphorylation of Akt1 (FIG. 4A) nor Akt3 (FIG. 4C) on cIEF electropherograms compared to the treatment of insulin alone. Interestingly, treatment of human preadipocytes with insulin together with cinnamaldehyde substantially increased pAkt2 phosphorylation compared to the treatment of insulin alone (FIG. 4B). Specifically, treatment with insulin together with cinnamaldehyde substantially increased pAkt (S473) phosphorylation compared to that of insulin alone (FIG. 4D). Consistent with cIEF immunoassays, capillary Western immunoassays further confirmed the synergy between insulin and cinnamaldehyde in substantially increased pAkt (Ser473) phosphorylation compared to insulin treatment alone (FIG. 4E). On average, treatment with both insulin and cinnamaldehyde increased pAkt (Ser473) phosphorylation by nearly 4 folds compared to treatment with insulin alone (FIG. 4F). Furthermore, comparison of the peaks attributable to pAkt2 (Ser473) on Akt2 electropherograms also revealed a nearly 4 folds increased in pAkt2 (Ser473) phosphorylation following treatment with insulin together cinnamaldehyde compared to insulin alone (FIGS. 4B and 4F).

### Example 2: Synergistic Phosphorylation and Enhanced Insulin-Stimulated Glucose Uptake Cinnamaldehyde and Curcumin Synergize to Promote pAkt (Thr450) Phosphorylation

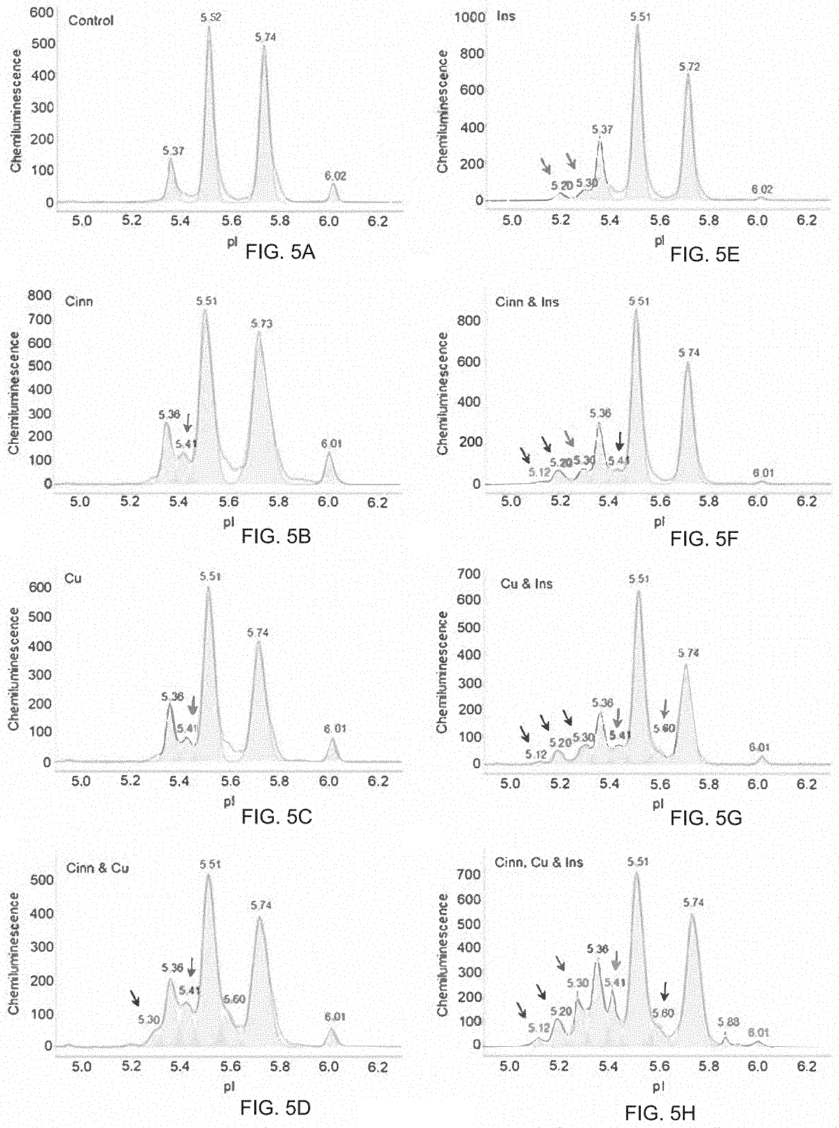

In addition to cinnamaldehyde, curcumin was identified as another insulin sensitizer (Table 1). Curcumin is a principal curcuminoid of turmeric. Like cinnamaldehyde, treatment of human primary preadipocytes with curcumin also induced a new peak at pI 5.41 on the Akt2 electropherogram (FIGS. 5A-5C). Interestingly, treatment with both cinnamaldehyde and curcumin substantially increased the intensity of the peak at pI 5.41 on the Akt2 electropherogram (FIG. 5D). Taken together, cinnamaldehyde and curcumin both individually and synergistically promoted the formation of the new peak at pI 5.41 on the Akt2 electropherogram, which was attributed to pAkt (Thr450).

TABLE 1
List of Insulin-Sensitizing Phytonutrients

| No | Phytonutrient | EC₅₀ |
|---|---|---|
| 1 | Cinnamaldehyde | 40 μM |
| 2 | Curcumin | 20 μM |

*Insulin-sensitizing phytonutrients were used at their half-maximal effective concentrations (EC₅₀) individually, or ⅕ of EC₅₀ values in combinations.

Cinnamaldehyde and Curcumin Synergize to Promote Insulin-Stimulated pAkt2 (Ser473) Phosphorylation.

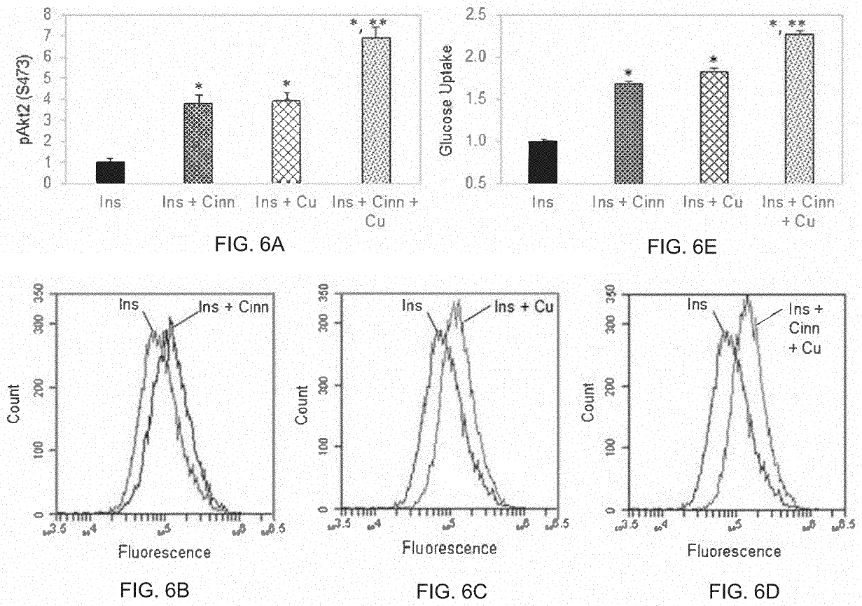

Similar to cinnamaldehyde, curcumin also promoted insulin-stimulated pAkt2 (Ser473) phosphorylation (FIGS. 5E-5G). Interestingly, curcumin synergized with cinnamaldehyde to further enhance insulin-stimulated pAkt2 (Ser473) phosphorylation (FIG. 51I). On the one hand, treatment with insulin together with either cinnamaldehyde or curcumin individually increased pAkt2 (Ser473) by approximately 4 folds (FIG. 6A). In contrast, treatment with insulin together with both cinnamaldehyde and curcumin increased pAkt2 (Ser473) by approximately 7 folds (FIG. 6A).

### Cinnamaldehyde and Curcumin Synergize to Enhance Insulin-Stimulated Glucose Uptake.

The synergy between curcumin and cinnamaldehyde to promote insulin-stimulated pAkt2 (Ser473) phosphorylation was further observed with glucose uptake by preadipocytes. The glucose uptake assay used 2-NBDG, a fluorescently-labeled deoxyglucose analog, as a probe for the detection of glucose taken up by human primary preadipocytes. Flow cytometry was used to detect 2-NBDG uptake in single preadipocytes. Insulin treatment together with either cinnamaldehyde or curcumin individually increased glucose uptake by preadipocytes by approximately 1.6 and 1.7 folds, respectively, compared to insulin treatment alone (FIGS. 6B, 6C, & 6E). Significantly, insulin treatment together with both cinnamaldehyde and curcumin increased glucose uptake by preadipocytes by approximately 2.3 folds (FIGS. 6D & 6E).

### Example 3: Identification and Rational Combination of Insulin-Sensitizing and Anti-Adipogenic Phytonutrients

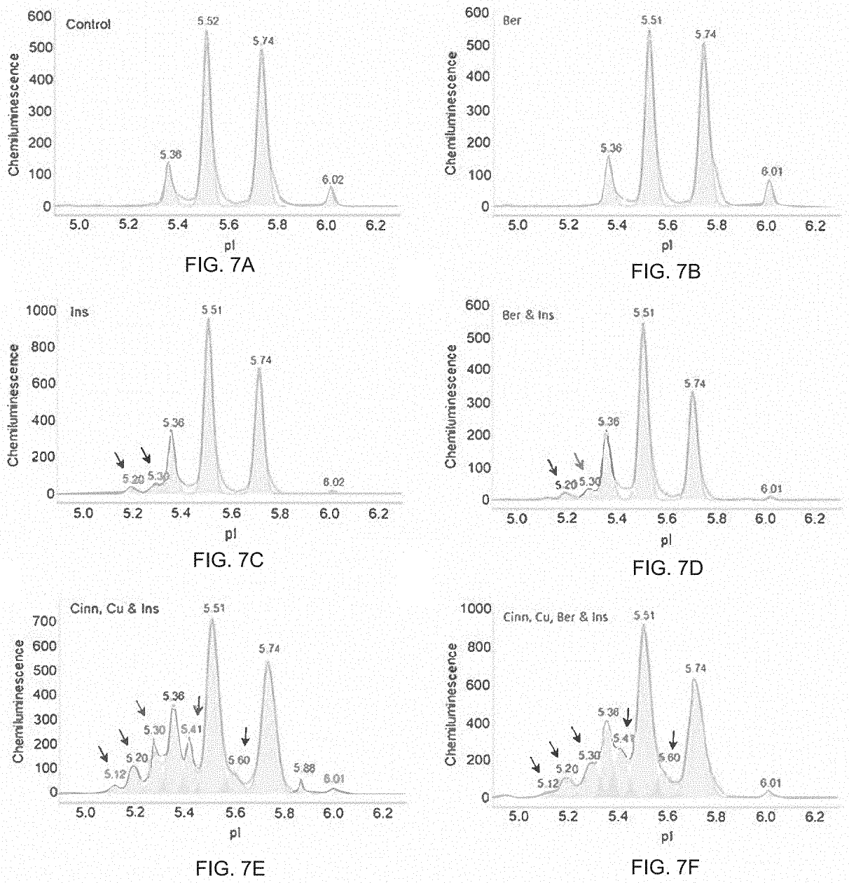

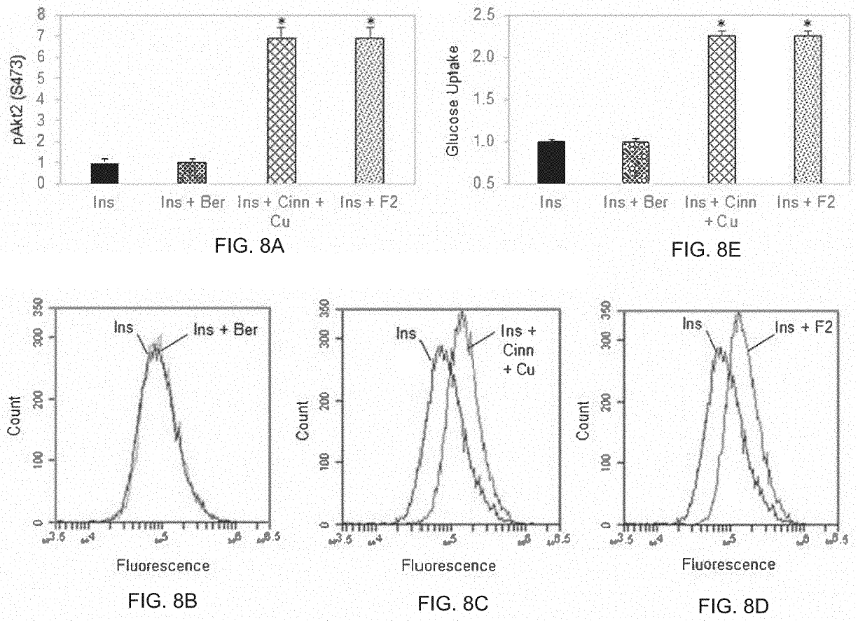

Nine anti-adipogenic and non-insulin sensitizing phytonutrients using human primary preadipocytes were identified (Table 2). The anti-adipogenecity of these phytonutrients was recognized. In addition, these phytonutrients also met the following selection criteria: (1) had no effect on pAkt (Thr450) phosphorylation; (2) did not interfere with the effects of cinnamaldehyde or curcumin on pAkt (Thr450) phosphorylation; (3) did not interfere with the effects of insulin-stimulated phosphorylation of pAkt2 (Ser473) or glucose uptake; and (4) did not interfere with the synergy between cinnamaldehyde, curcumin, and insulin on the promotion of pAkt2 (Ser473) phosphorylation or glucose uptake. FIGS. 7A-7F and FIGS. 8A-8E provide examples on the selection of berberine, an anti-adipogenic and non-insulin sensitizing phytonutrient that met all of the aforementioned criteria.

TABLE 2
List of Anti-Adipogenic Phytonutrients

| No | Phytonutrient | EC₅₀ | |
|---|---|---|---|
| 1 | Berberine | 10 | μM |
| 2 | Epigallocatechin Gallate | 20 | μM |
| 3 | Fucoidan | 100 | μg/ml |
| 4 | Fisetin | 50 | μM |
| 5 | Hesperidin | 20 | μM |
| 6 | Indole-3-Carbinol | 20 | μM |
| 7 | Luteolin | 20 | μM |
| 8 | Quercetin | 25 | μM |
| 9 | Resveratrol | 40 | μM |

*Anti-adipogenic phytonutrients were used at their half-maximal effective concentrations (EC₅₀) individually, or ⅕ of EC₅₀ values in combinations.

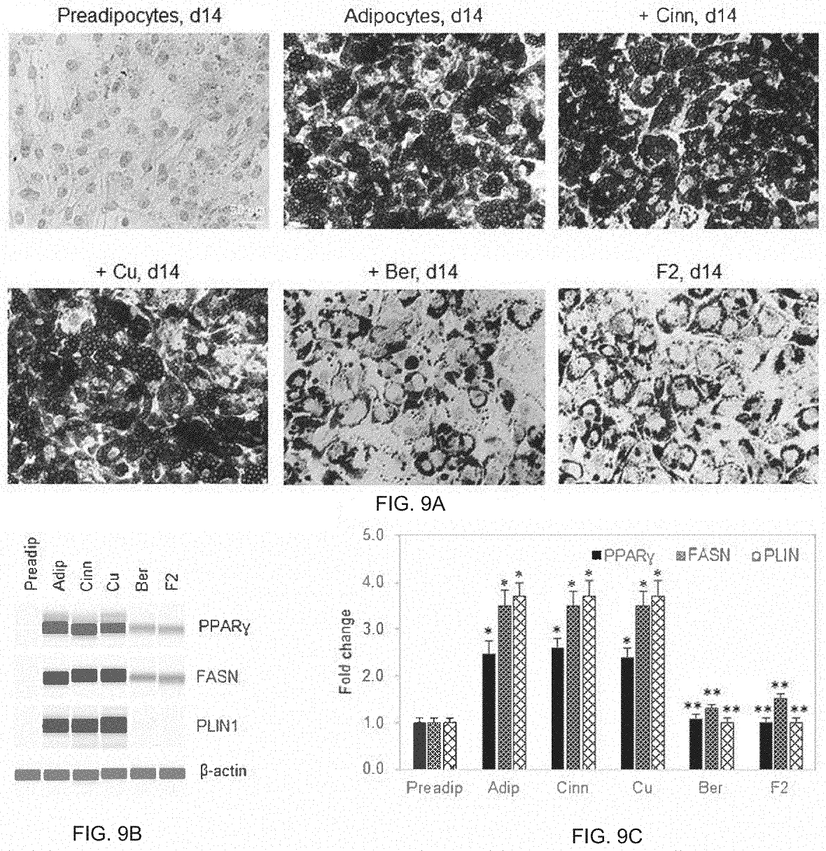

The combination of cinnamaldehyde, curcumin, and berberine, also known as F2 formulation, was capable of enhancing insulin sensitivity while inhibiting fat cell differentiation. Berberine, a strong inhibitor of adipogenesis, did not interfere with the insulin sensitizing capability of cinnamaldehyde or curcumin (FIGS. 7A-7F). Berberine by itself had no effect on Akt2 phosphorylation and did not interfere with the insulin-induced phosphorylation of Akt2 (FIGS. 7A-7D and FIG. 8A). As a component of F2 formulation, berberine did not interfere with the synergy between cinnamaldehyde and curcumin to enhance insulin-stimulated pAkt2 (Ser473) phosphorylation (FIGS. 7E, 7F and FIG. 8A). Furthermore, berberine had no effect on insulin-stimulated glucose uptake and did not interfere with the synergy between cinnamaldehyde and curcumin to enhance insulin-stimulated glucose uptake in preadipocytes (FIGS. 8B-8E). Significantly, as a component of the F2 formulation, berberine was mainly responsible for inhibiting intracellular lipid accumulation in differentiating adipocytes (FIG. 9A). Berberine was also mainly responsible for the inhibition of the expression of adipogenic biomarker proteins peroxisome proliferator-activated receptor y (PPARy), fatty acid synthase (FASN), and perilipin 1 (PLIN1) (FIGS. 9B and 9C). Taken together, the F2 formulation was capable of both promoting insulin-stimulated glucose uptake and inhibiting adipogenesis.

### Example 4: F2 Prevented Weight Gain in a Diet-Induced Obesity (DIO) Mouse Model

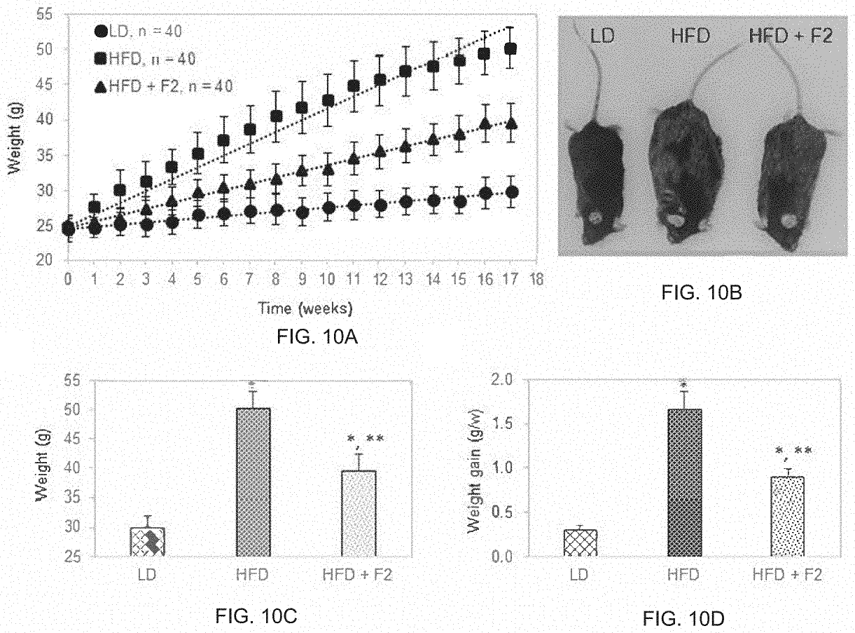

The therapeutic effects of F2 for obesity prevention were evaluated using a DIO mouse model (FIGS. 10A-10D). Mice were divided into three groups: a group of 40 mice were fed with a lean diet, a group of 40 mice were fed with a high-fat diet, and a group of 40 mice were fed with a high-fat diet supplemented with F2. F2 was supplemented to ground pellets at 0.1% by weight, leading to an approximately daily dose of 200 mg/kg for mice, or approximately 16 mg/kg of human equivalent dose. All mice were placed on their respective diets for 17 weeks. At the beginning of the experiments, all mice were male, approximately 10 weeks old, and had bodyweights of approximately 25 grams. Mice fed with a lean diet gained weight at a steady rate of 0.3 gram per week and reached an average of 30 grams in bodyweight after 17 weeks. Mice fed with a high-fat diet gained weight rapidly at a rate of 1.7 grams per week and reached an average of 50 grams in bodyweight after 17 weeks. Interestingly, mice fed with a high-fat diet supplemented with F2 gained weight a rate of 0.9 grams per week and reached an average of 40 grams in bodyweight after 17 weeks. On average, F2 supplementation reduced weight gain of DIO mice by approximately 50%.

### Example 5: F2 Improved Glucose Tolerance and Reduced HbAlc in DIO Mice

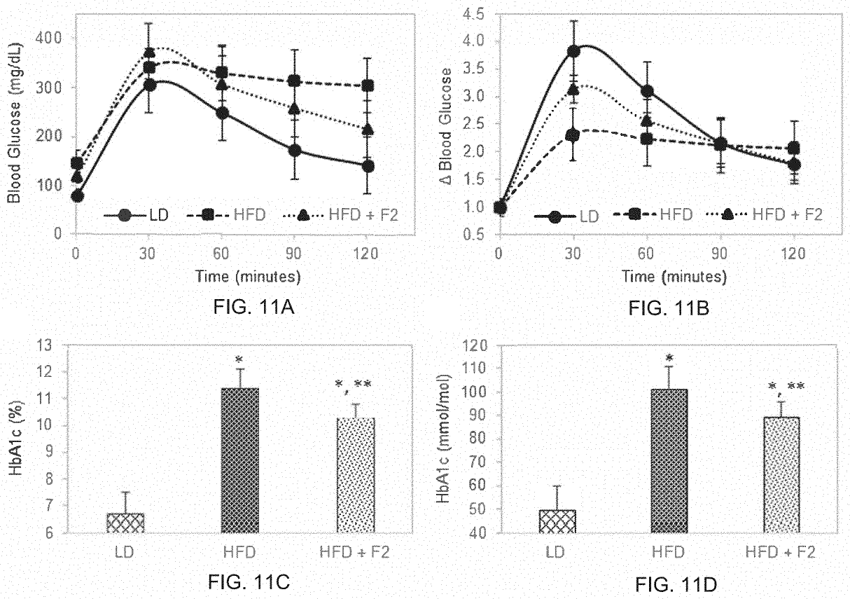

At 16^{th} week, glucose tolerance tests were performed for all mice after 16 hours of overnight fasting (FIGS. 11A & 11B). Fasting blood glucose levels were 80 mg/dL, 147 mg/dL, and 119 mg/dL for mice fed with a lean diet, a high-fat diet, and a high-fat diet supplemented with F2, respectively. Following intraperitoneal injection of 20% glucose at 2 g of glucose per kg of body mass, blood samples were collected via the tail veins at 0, 30, 60, 90, and 120 minutes and measured for blood glucose levels using a glucometer. Mice fed with a lean diet showed strong ability to dynamically regulate blood glucose level. In mice fed with a lean diet, blood glucose increased by nearly 4 folds, peaked at around 30 minutes post-injection, and steadily declined to slightly less than 2 folds higher than the baseline blood glucose level at 120 minutes post-injection. In contrast, mice fed with a high-fat diet were unable to regulate blood glucose level. In mice fed with a high-fat diet, blood glucose increased by 2.3 folds at 30 minutes post-injection, and stayed elevated at above 2.0 folds higher than the baseline blood glucose level until 120 minutes post-injection. Interestingly, mice fed with a high-fat diet supplemented with F2 had better control of blood glucose level compared to mice fed with a high-fat diet alone. Following glucose injection, blood glucose increased by more than 3 folds at 30 minutes post-injection, and steadily declined to less than 2 folds higher than the baseline blood glucose level at 120 minutes post-injection. Furthermore, HbAlc levels measured in terminally collected blood samples at 17^{th} week were 6.7% or 50 mmol/mol for mice in the lean diet group, 11.4% or 101 mmol/mol for mice in the high-fat diet group, and 10.3% or 89 mmol/mol in the high-fat diet supplemented with F2 group (FIGS. 11C and 11D). Taken together, F2 improved glucose tolerance and reduced HbAlc in DIO mice.

### Example 6: F2 Reduced Blood Lipids in DIO Mice

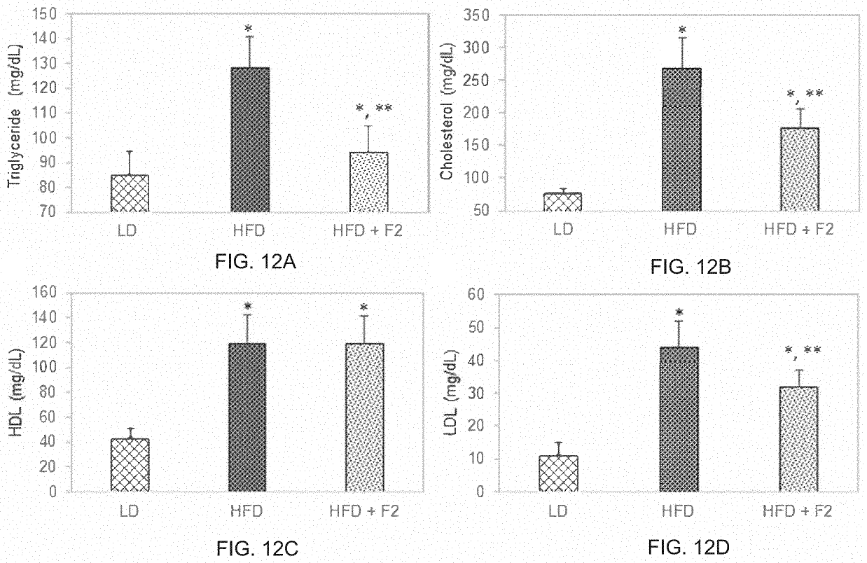

At 17^{th} week, blood and tissue samples were terminally collected from all mice and measured for triglyceride, total cholesterol, high-density lipoprotein (HDL) cholesterol, and low-density lipoprotein (LDL) cholesterol. The average triglyceride levels were 85 mg/dL, 128 mg/dL, and 94 mg/dL for mice fed with a lean diet, a high-fat diet, and a high-fat diet supplemented with F2, respectively (FIG. 12A). The average total cholesterol levels were 77 mg/dL, 269 mg/dL, and 176 mg/dL for mice fed with a lean diet, a high-fat diet, and a high-fat diet supplemented with F2, respectively (FIG. 12B). The average HDL cholesterol levels were 43 mg/dL, 119 mg/dL, and 119 mg/dL for mice fed with a lean diet, a high-fat diet, and a high-fat diet supplemented with F2, respectively (FIG. 12C). The average LDL cholesterol levels were 11 mg/dL, 44 mg/dL, and 32 mg/dL for mice fed with a lean diet, a high-fat diet, and a high-fat diet supplemented with F2, respectively (FIG. 12B). The blood lipid profiles indicated that F2 supplementation reduced blood triglyceride and LDL cholesterol in DIO mice.

### Example 7: F2 Reduced Visceral Adiposity and Liver Steatosis in DIO Mice

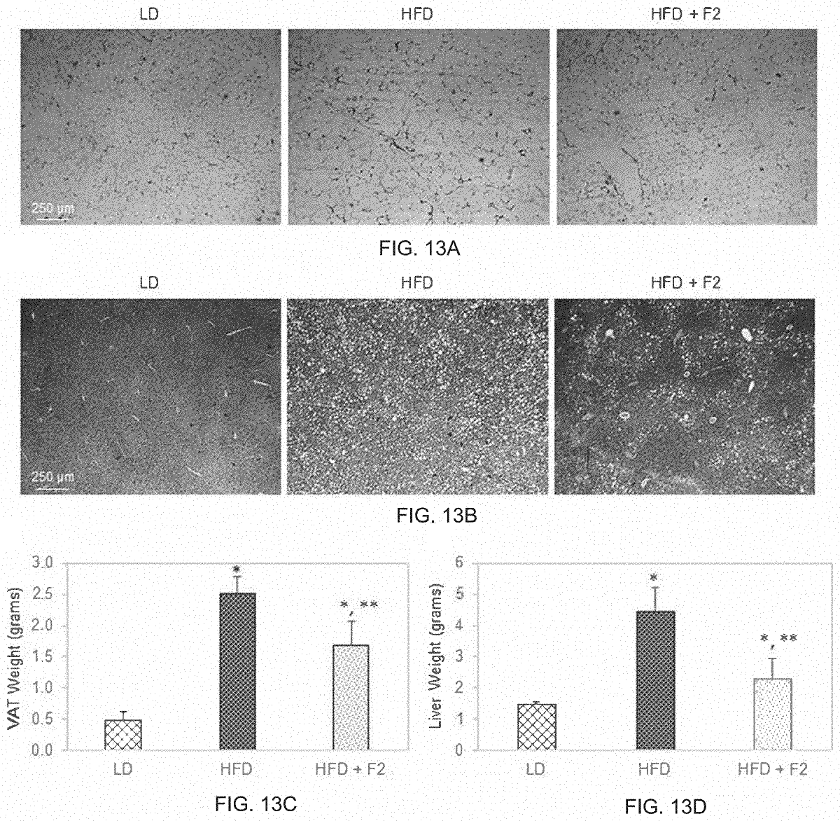

Terminally collected liver and visceral adipose tissues were sent to IHC World (Woodstock, Md.) for hematoxylin & eosin (H&E) histology preparation. H&E histology revealed an average diameter of lipid droplets of visceral adipocytes that was 2 times higher for mice fed with a high-fat diet compared to those fed with a lean diet (FIG. 13A). In contrast, the average diameter of lipid droplets of visceral adipocytes were 1.5 times higher for mice fed with a high-fat diet supplemented with F2 compared to those fed with a lean diet. Consistently, the average VAT weights were 0.5 g, 2.5 g, and 1.7 g for mice fed with a lean diet, a high-fat diet, and a high-fat diet supplemented with F2, respectively (FIG. 13C). Furthermore, H&E histology revealed a complete absence of any lipid droplet accumulation in liver tissues of mice fed with a lean diet, severe lipid droplet accumulation in liver tissues of mice fed with a high-fat diet, and substantially reduced level of lipid droplet accumulation in liver tissues of mice fed with a high-fat diet supplemented with F2 compared to those of mice fed with a high-fat diet alone (FIG. 13B). On average, the liver weights were approximately 1.5 g, 4.5 g, and 2.3 g for mice fed with a lean diet, a high-fat diet, and a high-fat diet supplemented with F2, respectively (FIG. 13D). Taken together, F2 supplementation reduced liver steatosis and visceral adiposity in DIO mice.

### SUMMARY

Using nanofluidic proteomics, phytonutrients were classified as insulin sensitizing or anti-adipogenic based on their ability to enhance insulin-stimulated Akt2 phosphorylation and glucose uptake or inhibit adipogenesis, respectively. In one aspect, insulin sensitizing phytonutrients (Table 1) were identified by their unique ability to induce the formation of a new peak at pI 5.41 on the Akt2 cIEF electropherogram, which was attributed to increased pAkt2 (Thr450) phosphorylation. In general, phosphorylation of pAkt2 (Thr450) primes Akt2 for insulin-stimulated phosphorylation at pAkt2 (Thr308) and pAkt2 (Ser473), which fully activates Akt2 [10]. Insulin-sensitizing phytonutrients cinnamaldehyde and curcumin synergized to increase the peak intensity at pI 5.41 on Akt2 cIEF electropherogram, which indicates increased pAkt2 (Thr450) phosphorylation. In addition, cinnamaldehyde and curcumin synergized to enhance insulin-stimulated phosphorylation at pAkt2 (Ser473) by nearly 7 folds and glucose uptake by 2.3 folds compared to insulin alone. In another aspect, anti-adipogenic phytonutrients are non-insulin sensitizing and strong suppressors of adipogenesis (Table 2). Anti-adipogenic phytonutrients did not interfere with insulin signaling or the activities of insulin-sensitizing phytonutrients. In human primary preadipocytes, a combination of insulin-sensitizing and anti-adipogenic phytonutrients comprising cinnamaldehyde, curcumin, and berberine was capable of enhancing glucose uptake while inhibiting adipogenesis. The combination of cinnamaldehyde, curcumin, and berberine was named F2 formulation. In an animal model of diet-induced obesity and diabetes, dietary supplementation with F2 formulation improved glucose tolerance, reduced HbAlc, prevented weight gain, reduced blood lipid level, and suppressed liver steatosis. In summary, a proteomic method was used to identify insulin-sensitizing phytonutrients and a platform for the rational combinations of insulin-sensitizing and anti-diabetic phytonutrients. Furthermore, the therapeutic potential for the F2 formulation to provide glycemic control without weight gain for long-term diabetes care was demonstrated.

### Materials and Methods

Human Primary Preadipocytes. Primary human preadipocytes were isolated from omental adipose tissues overweight and diabetic donors who were undergoing elective surgery using a previously published protocol [11]. Primary human preadipocytes were maintained in growth media comprising Minimum Essential Medium a supplemented with 10% fetal bovine serum, 100 units/ml penicillin, and 100 μg/ml streptomycin.

Screening for Insulin-Sensitizing Phytonutrients. Primary human preadipocytes were treated for 30 minutes with 100 nM insulin (Humulin R, Eli Lilly, Indianapolis, Ind.), individual phytonutrients (Table 1 & Table 2), or combinations of insulin and phytonutrients. Total cell extracts (TCE) were collected and subjected to nanofluidic proteomic analysis including capillary isoelectric focusing (cIEF) immunoassays and capillary Western (CW) immunoassays.

Preparation of Total Cell Extracts. Approximately one million cells were incubated on ice for 10 minutes with 60 μl of lysis buffer (cat. no. 040-764, ProteinSimple, Santa Clara, Calif., USA), sonicated 4 times for 5 seconds each, mixed by rotation for 2 hours at 4° C., and centrifuged at 12,000 rpm in an Eppendorf 5430R microfuge for 20 minutes at 4° C. The supernatant was collected as the cell lysate. The total protein concentration in the cell lysate was determined with a Bradford protein assay and adjusted to a final concentration of 0.3 μg/μl with separation gradients (cat. no. Premix G2, pH 5-8, ProteinSimple, Santa Clara, Calif.) for charge-based cIEF immunoassays or to 0.4 μg/μl with denaturing buffers (cat. no. PS-ST01EZ or PS-ST03EZ, ProteinSimple) for size-based Western immunoassays.

Capillary Isoelectric Focusing Immunoassays. Cell lysates in separation gradients were loaded into 384-well assay plates (cat. no. 040-663, ProteinSimple) preloaded with primary and secondary antibodies and chemiluminescent substrates. Charge-based protein separation and detection in individual capillaries were performed using the default protocols of the NanoPro 1000 system (ProteinSimple). Hsp70 was used as the loading control. All cIEF immunoassays were performed in triplicate for each protein, and duplicate experiments were performed for each treatment condition, producing six repeated measurements per protein.

Capillary Western Immunoassays. Cell lysates in denaturing buffers were denatured at 95° C. for 5 minutes, and then transferred to assay plates (cat. no. SM-W004 or SM-W008, ProteinSimple) preloaded with blocking reagents, wash buffer, primary and secondary antibodies, and chemiluminescent substrates. Sized-based protein separation and detection in capillaries were performed using the default protocols of the Jess system (ProteinSimple). β-Actin was used as loading controls. All capillary Western immunoassays were performed in triplicate for each protein, and duplicate experiments were performed for each treatment condition, producing six repeated measurements per protein.

Glucose Uptake Assays. Primary human preadipocytes grown to 70% confluence in growth media, washed with phosphate buffered saline, and replaced with glucose-free DMEM media. Following 2 hours of incubation in glucose-free DMEM media, preadipocytes were treated with 100 μg/ml of 2-NDBG fluorescent glucose analog and insulin (100 nM) or phytonutrients (EC₅₀) for 30 minutes. Preadipocytes were collected and analyzed using the Acuri C6 flow cytometer (BD Biosciences, San Jose, Calif.) and the 485 nm excitation and 535 nm emission filters.

Adipogenesis Assays. Primary human preadipocytes were grown to confluence in growth media. At 2 days post-confluence, growth media were aspirated off the culture dishes and complete differentiation media were added. Complete differentiation media comprise DMEM/F22 with 18.5 mM glucose, HEPES (15 mM), NaHCO3 (25 mM), 100 units/ml penicillin, 100 μg/ml streptomycin, d-biotin (33 μM), pantothenate (17 μM), dexamethasone (100 nM), insulin (100 nM), rosiglitazone (1 μM), IBMX (0.5 mM), triiodothyronine (T3, 2 nM), and transferrin (10 μg/ml). On day three post-differentiation, complete differentiation media were replenished. On day seventh post-differentiation, complete differentiation media were replaced with maintenance media. Maintenance media comprise DMEM/F22, 100 units/ml penicillin, 100 μg/ml streptomycin, HEPES (15 mM), NaHCO3 (25 mM), d-biotin, pantothenate, insulin (10 nM), and dexamethasone (10 nM). Maintenance media were replenished on days tenth post-differentiation. Complete differentiation of preadipocytes into adipocytes were achieved on day fourteenth post-differentiation. To screen for anti-adipogenic phytonutrients, individual phytonutrients were mixed in complete differentiation media from day 0 to day 6^{th} post-differentiation. Phytonutrients were removed together with complete differentiation media on day 7^{th} post-differentiation following the replacement with maintenance media. Anti-adipogenic phytonutrients were identified based on their ability to: (1) suppress the expression of adipogenic biomarkers using proteomic assays of total cell extracts collected on day 6^{th} post-differentiation, and (2) suppress intracellular lipid droplet accumulation using Oil Red O staining assays on day 14^{th} post-differentiation.

Antibodies. The antibodies used are listed in Table 3.

TABLE 3
List of Primary and Secondary Antibodies

| No | Antibody | Cat. No. | Vendor |
|---|---|---|---|
| 1 | pan-Akt | 8312 | Santa Cruz Biotech |
| | | | (Dallas, TX) |
| 2 | Akt1 | 2938 | Cell Signaling (Danvers, |
| | | | MA) |
| 3 | Akt2 | 3063 | Cell Signaling |
| 4 | Akt3 | 8018 | Cell Signaling |
| 5 | pAkt (Thr308) | 9275 | Cell Signaling |
| 6 | pAkt (Thr450) | 9267 | Cell Signaling |
| 7 | pAkt (Ser473) | 4060 | Cell Signaling |
| 8 | PPARγ | CS2443 | Cell Signaling (Danvers, |
| | | | MA) |
| 9 | FASN | CS3189 | Cell Signaling |
| 10 | PLIN1 | CS9349 | Cell Signaling |
| 11 | β-actin | MAB8929 | R&D Systems (Minneapolis, |
| 12 | HSP70 | 4872 | Cell Signaling |
| 13 | Secondary antibody | 040-656 | Protein Simple (Santa |
| | (anti-rabbit | | Clara, CA) |
| 14 | Secondary antibody | 042-206 | Protein Simple |
| | (anti-rabbit | | |
| 15 | Secondary antibody | 042-205 | Protein Simple |
| | (anti-mouse | | |
| 16 | Secondary antibody | 043-819 | Protein Simple |
| | (anti-rabbit | | |
| 17 | Secondary antibody | 043-821 | Protein Simple |
| | (anti-mouse | | |

F2 Formulation. F2 formulation is a combination of three phytonutrients cinnamaldehyde, curcumin, and berberine. For tissue cultures, F2 formulation is the combination of cinnamaldehyde, curcumin, and berberine at the final concentrations of 8 μM, 4 μM, and 2 μM, respectively. For animal studies, F2 formulation is the combination of cinnamaldehyde, curcumin, and berberine at 1:1:1 weight ratio. F2 is supplemented to the diet at 0.1% by weight, leading to an approximately daily dose of 200 mg/kg for mice, or approximately 16 mg/kg of human equivalent dose.

DIO Animal Model. C57BL/6J mice (male, —10 weeks old, Jackson Lab, Bar Harbor, Me.) were divided into three groups: a group of 40 mice fed with a lean diet, a group of 40 mice fed with a high-fat diet, and a group comprising mice fed with a high-fat diet supplemented with F2 formulation. The lean diet (cat. no. TD7001, Teklad Diets, Madison, Wis.) comprised protein (25.2% by weight), carbohydrate (39.5% by weight), fat (4.4% by weight), and others (30.9% by weight, ash, fibers, others). The lean diet has 3 kcal/g, with 34% of kcal from protein, 53% of kcal from carbohydrate, and 13% of kcal from fat. The high-fat diet (cat. no. TD88137, Teklad Diets) comprised protein (17.3% by weight), carbohydrate (48.5% by weight), fat (21.2% by weight), and others (13% by weight, ash, fibers, others). The high fat diet has 4.5 g/kcal, with 15.2% of kcal from protein, 42.7% of kcal from carbohydrate, and 42% of kcal from fat. Mice groups were placed on their respective diets in the form of ground pellets for 17 weeks. Glucose tolerance tests using standard protocols were performed at 16^{th} week. Terminal tissue and blood samples collection were performed at 17^{th} week. Collected liver and visceral adipose tissues were sent to IHC WORLD (Woodstock, Md.) for histopathology analysis. Collected blood samples were analyzed for HbAlc, triglyceride, cholesterol, HDL, and LDL using commercially available assay kits (Table 4). All animal studies were performed in conformity with the Public Health Service Policy on Humane Care and Use of Laboratory Animals and with the approval of the Animal Care and Use Committee at Roseman University of Health Sciences.

TABLE 4
List of Biochemical Assays

| No | Biochemical Assay | Cat. No. | Vendor |
|---|---|---|---|
| 1 | Glucose Uptake | 600470 | Cayman Chem (Ann Arbor, MI) |
| 2 | HbA1c | LS-F36432 | LS Bio (Seattle, WA) |
| 3 | Triglyceride | Ab65336 | Abcam (Cambridge, MA) |
| 4 | Cholesterol | Ab65359 | Abcam |
| 5 | HDL | Ab65390 | Abcam |
| 6 | VLDL/LDL | Ab65390 | Abcam |

### REFERENCES

1. Centers for Disease Control and Prevention. National Diabetes Statistics Report 2020: Estimates of Diabetes and Its Burden in the United States.
2. American Diabetes Association. Diagnosis and classification of diabetes mellitus. Diabetes Care 2013, 36 Suppl 1, S67-74.
3. Katsarou, A.; Gudbjornsdottir, S.; Rawshani, A.; Dabelea, D.; Bonifacio, E.; Anderson, B. J.; Jacobsen, L. M.; Schatz, D. A.; Lernmark, A. Type 1 diabetes mellitus. Nat Rev Dis Primers 2017, 3, 17016.
4. Van Gaal, L.; Scheen, A. Weight management in type 2 diabetes: current and emerging approaches to treatment. Diabetes Care 2015, 38, 1161-1172.
5. Kusminski, C. M.; Bickel, P. E.; Scherer, P. E. Targeting adipose tissue in the treatment of obesity-associated diabetes. Nat Rev Drug Discov 2016, 15, 639-660.
6. Martel, J.; Ojcius, D. M.; Chang, C. J.; Lin, C. S.; Lu, C. C.; Ko, Y. F.; Tseng, S. F.; Lai, H. C.; Young, J. D. Anti-obesogenic and antidiabetic effects of plants and mushrooms. Nat Rev Endocrinol 2017, 13, 149-160.
7. Tokarz, V. L.; MacDonald, P. E.; Klip, A. The cell biology of systemic insulin function. J Cell Biol 2018, 217, 2273-2289.
8. Gonzalez, E.; McGraw, T. E. The Akt kinases: isoform specificity in metabolism and cancer. Cell Cycle 2009, 8, 2502-2508.
9. Urasaki, Y.; Beaumont, C.; Talbot, J. N.; Hill, D. K.; Le, T. T. Akt3 Regulates the Tissue-Specific Response to Copaiba Essential Oil. Int J Mol Sci 2020, 21.
10. Bellacosa, A.; Chan, T. O.; Ahmed, N. N.; Datta, K.; Malstrom, S.; Stokoe, D.; McCormick, F.; Feng, J.; Tsichlis, P. Akt activation by growth factors is a multiple-step process: the role of the PH domain. Oncogene 1998, 17, 313-325.
11. Lee, M. J.; Fried, S. K. Optimal protocol for the differentiation and metabolic analysis of human adipose stromal cells. Methods Enzymol 2014, 538, 49-65.

1. A composition comprising a combination of at least two phytonutrients selected from the group consisting of cinnamaldehyde, curcumin, and berberine.

2. The composition of claim 1, wherein said combination of phytonutrients comprises cinnamaldehyde and curcumin.

3. The composition of claim 1, wherein said combination of phytonutrients comprises cinnamaldehyde and berberine.

4. The composition of claim 1, wherein said combination of phytonutrients comprises cinnamaldehyde, curcumin, and berberine.

5. A method of treating or preventing diabetes comprising administering a therapeutically effective amount of the composition of claim 1 to a subject in need thereof.

6. The method of claim 5, wherein said method improves glucose tolerance, reduces HbAlc, prevents weight gain, reduces blood lipid level, or suppresses liver steatosis.

7. The method of claim 6, wherein said method improves glucose tolerance.

8. The method of claim 6, wherein said method reduces HbAlc.

9. The method of claim 6, wherein said method prevents weight gain.

10. The method of claim 6, wherein said method reduced blood lipid level.

11. The method of claim 6, wherein said method suppresses liver steatosis.
